(19) United States
(12) Patent Application Publication
(10) Pub. No.: US 2009/0169485 A1
(43) Pub. Date: Jul. 2, 2009

(54) Gene therapy and pharmaceutical composition for prevention and treatment of lung cancer

(75) Inventors: Myung-Haing Cho, Seoul (KR); Chong-su Cho, Seoul (KR)

Correspondence Address: BIRCH STEWART KOLASCH & BIRCH, PO BOX 747, FALLS CHURCH, VA, 22040-0747, US

(73) Assignee: SEOUL NATIONAL UNIVERSITY INDUSTRY FOUNDATION

(21) Appl. No.: 12/285,867

(22) Filed: Oct. 15, 2008

(30) Foreign Application Priority Data
Dec. 26, 2007 (KR) 10-2007-0137115

(51) Int. Cl. A61K 9/12; A61K 31/7088; A61K 38/00; A61K 9/127; A61P 35/04; A61K 9/16; A61K 35/76

(52) U.S. Cl. 424/45; 514/44; 514/2; 424/450; 424/489; 424/93.6

(57) ABSTRACT

Disclosed herein is a gene therapeutic agent and pharmaceutical composition for the prevention and treatment of lung cancer. For aerosol delivery, chemically synthesized polyester amine is used as a carrier in the gene therapeutic agent. The polyester amine/Akt1 siRNA complex is found to be effectively delivered to the lungs of K-ras null mice through a nose-only inhalation system and to significantly suppress lung cancer progression as denoted by gene delivery efficiency and inhibition of Akt-related signals and cell cycle. Thus, the aerosol delivery of polyester amine-mediated Akt1 siRNA is provided as an effective model for noninvasive gene therapy.

### BACKGROUND OF THE INVENTION

1. Field of the Invention

The present invention relates to gene therapeutic agents and pharmaceutical compositions for the prevention and treatment of lung cancer.

2. Description of the Related Art

The lungs have the benefit of easy in vivo access and thus are a relatively accessible target for gene therapy using local application of a gene delivery system. In the field of pulmonary medicine, extensive attempts have been made to locate non-invasive therapies targeting-diverse lung disorders including cancer (Dailey et al., 2004; Ferrari et al., 2002; Merdan et al., 2002).

The gene therapy approach to lung disorder, including intravenous injection and administration via a nasal and other catheters were conducted in various animal models (Gautam et al., 2001; Goula et al., 2000). However, such a gene transfer strategy is invasive or might be unsuitable for use in the effective delivery of genes of interest to lung tissues.

A number of vector systems, both viral and non-viral, have been employed for gene delivery to the lung. Thanks to the ability thereof to infect cells and promote the expression of genes of interest therein, viral vectors are widely used. However, various factors such as immune response following repeated administration, difficulty in large-scale production, etc., restrict the practical use of viral vectors (Densmore, 2006).

Having various advantages including easy manipulation, low cost, higher safety and less immunogenicity over viral vectors, non-viral vectors have continued to attract extensive attention. Various non-viral vectors were developed to transfer genes to various target organs.

Aerosol gene delivery, representative of a non-invasive approach to lung disorder, is found to effectively transfer genes of interest to the target organ (Merlin et al., 2001). In this regard, the present inventors previously disclosed that polyester amine may be a promising non-invasive alternative for effective gene delivery thanks to its degradability, ability to form a complex with DNA, low toxicity, and improved gene transfer efficiency (Park et al., 2005).

The serine-threonine Akt kinase (also known as protein kinase B) is an important regulator of cell survival and cell proliferation (Vivano and Sawyers, 2002). Akt plays an essential role in cancer by stimulating cell proliferation and inhibiting apoptosis (Lawlor and Alessi, 2001). Furthermore, amplification of genes encoding Akt isoforms has been found in many tumors (Vivano and Sawyers, 2002).

Dominant negative alleles of Akt have been reported to block cell survival and to induce an apoptotic response (Li et al., 1998). Because Akt promotes both cell survival and proliferation, specific inhibition of its downstream signaling pathway by expression of an Akt mutant, for instance, is a rational therapeutic strategy for tumors showing amplification of the Akt gene.

Approximately 30% of human tumors carry ras gene mutations. Of the three members of ras family (K-ras, N-ras and H-ras) K-ras are found to be the most frequently mutated members in human tumors, including lung adenocarcinomas (25-50%) (Pellegata et al., 1996).

Mice carrying such mutations are highly predisposed to a range of tumor types and exhibit short latency and high penetration (Johnson et al., 2001). In the following Examples, K-ras null mice, a laboratory animal model of non-small cell lung cancer (NSLC) was used for in vivo assessment of the role of aerosol-delivered Akt1 siRNA in lung tumorigenesis.

siRNA induces a post-translational gene-silencing mechanism, causing degradation of mRNAs of the target gene homologous in sequence to the double strands of the siRNA (Hu et al., 2002; Cogoni and Macino, 2000).

When introduced into mammalian cells, chemically synthesized siRNAs can induce gene silencing therein. Transfection of short 21-nt RNA duplexes into mammalian cells interferes with gene expression and does not induce the unspecific anti-viral response (Elbashir et al., 2001).

Chemical synthesized siRNA usually induce only a transient reduction of endogenously expressed target mRNA. The development of siRNA technology opens the possibility of effective gene manipulation both in vitro and in vivo.

However, the successful in vivo application of siRNA depends on the use of a suitable delivery carrier of siRNA

Most conventional therapies are found not to be suitable for the treatment of lung cancer, and so there is a need for a promising novel therapeutic technology for treating lung cancer. The perception of the present inventors on the non-invasive delivery of Akt1 siRNA to the target leads to the present invention.

In recent years, much effort has focused on the development of aerosol gene delivery technology for the treatment of diverse lung diseases including cancer. This effort has involved finding appropriate non-viral DNA delivery carriers that both withstand the sheering force of nebulization and also function optimally in the lungs (Tehrani et al., 2007)

To enhance the biocompatibility of PEI for use as a non-viral vector, Ahn and colleagues synthesized a PEI derivative such as PEI-PEG copolymer (Ahn et al., 2002); however, the transfection efficiency of the copolymer was very low compared to PEI 25K, although the copolymer was degradable and less toxic.

With the aim of overcoming the problems encountered in the above example and many previous unsatisfactory efforts for the synthesis of PEI derivatives, the present inventors were prompted to synthesize a novel copolymer as new gene carrier, producing new degradable poly(ester amine) copolymer with high transfection efficiency and low toxicity (Park et al., 2005).

The Akt family is composed of three isoforms (Akt1, Akt2, and Akt3), which, in general, are broadly expressed, although there are some isoform-specific features (Vivance and Sawyer, 2002). In fact, Akt1 is implicated in the treatment resistance of NSLC, suggesting that Akt1 inhibition is a key factor specific for inducing cancer cell death (Brognard et al., 2001).

Although Akt1 activation has been implicated in up-regulated cell proliferation, in vivo effects of Akt1 in terms of cell proliferation and critical downstream effectors, such as mTOR, p70S6K, and 4E-BP1, remain largely uncertain.

As such, the present inventors undertook investigation into the function of Akt1 in lung cancer progression with the aid of Akt1 siRNA.

### SUMMARY OF THE INVENTION

Accordingly, the present invention has been made keeping in mind the above problems occurring in the prior art, and an object of the present invention is to provide an aerosol-deliverable, novel gene therapeutic agent for the prevention and treatment of lung cancer, which is superior in therapeutic efficiency to conventional gene therapy, thus ensuring the treatment efficiency survival of lung cancer patients for a longer time period.

It is another object of the present invention is to provide a pharmaceutical composition for inducing apoptosis in cancer cells, comprising an effector against Akt1.

In accordance with an aspect thereof, the present invention provides a gene therapeutic agent for prevention or treatment of lung cancer, comprising an effector for suppressing Akt1 activity, and a gene carrier, by being brought into contact with pulmonary tumor cells, wherein the effector is selected from a group consisting of siRNA, an antisense molecule, an antagonist, a ribozyme, an inhibitor, a peptide and a small molecule; the gene carrier is polyester amine; and the contact is conducted with the aid of a mean selected from a group consisting of a liposome, a nanoliposome, a ceramide-containing nanoliposome, a proteoliposome, a nanoparticulate, a calcium phosphor-silicate nanoparticulate, a calcium phosphate nanoparticulate, a silicon dioxide nanoparticulate, a nanocrystalline particulate, a semiconductor nanoparticulate, poly(D-arginine), a nanodendrimer, a virus, calcium phosphate nucleotide-mediated nucleotide delivery, electroporation, microinjection, and aerosol delivery.

In accordance with another aspect thereof, the present invention provides a pharmaceutical composition for prevention and treatment of lung cancer, comprising the gene therapeutic agent as an active ingredient, and a carrier.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

The above and other objects, features and other advantages of the present invention will be more clearly understood from the following detailed description taken in conjunction with the accompanying drawings, in which:

FIG. 1 is a photograph (×200) showing the delivery efficiency of poly(ester amine) as a gene carrier;

FIG. 2 shows the activity of Akt1 in the lungs of K-ras null mice exposed to the aerosol-delivered Akt1 siRNA, through Western blot analysis of the Akt family in a photograph (×200) (a), through densitometric analysis for bands of interest in graphs (b), through Western blot analysis of fluorescent-Akt proteins in a photograph (×200) (c), through densitometric analysis for the fluorescent-Akt proteins in graphs (d), through immunohistochemical analysis for the expression of fluorescent-Akt in the lungs of K-ras null mice in photographs (×200) (e), and in terms of fluorescent-Akt labeling index in the lungs of K-ras null mice in graphs (f) where CON stands for control, SCR for scrambled control and siAkt1 for Akt1 siRNA, and scale bars represent 100 μm;

FIG. 3 shows tumor pathologies of the lungs of K-ras null mice in photographs (×200), specifying injuries generated in the lungs (a), and histologic characteristics of the lungs (b), wherein lung adenocarcinoma is indicated by the arrows and dashed circles, CON stands for control, SCR for scrambled control and siAkt1 for Akt1 siRNA, and the scale bars represent 100 μm;

FIG. 4 shows Western blot analysis for Akt1-related proteins, mTOR, p70S6K and 4E-BP1 in the lungs of K-ras null mice exposed to aerosol-delivered Akt1 siRNA in a photograph (×200) (a) and densitometric analysis of bands of interest in graphs where CON stands for control, SCR for scrambled control, and siAkt1 for Akt1 siRNA (b); and

FIG. 5 shows the expression of proteins important for cell cycle regulation in the lungs of K-ras null mice exposed to aerosol-delivered Akt1 siRNA through Western blot analysis for cyclin D1, cyclin D3, CDK4 and PCNA in a photograph (×200) (a), through a densitometric analysis for bands of interest in graphs (b), through immunohistochemical analysis for PCNA in photographs (c) where dark brown colors indicate the expression of PCNA, and in terms of PCNA labeling index of the immunopositive cells in a graph (d) where the level of PCNA positive staining was determined by counting 3 randomly chosen fields per section and determining the percentage of DAB-positive cells per 100 cells at magnification times 400, and CON stands for control, SCR for scrambled control and siAkt1 for Akt1 siRNA.

### DESCRIPTION OF THE PREFERRED EMBODIMENTS

In accordance with an aspect thereof, the present invention pertains to a gene therapeutic agent for the prevention or treatment of lung cancer, comprising an effector for suppressing Akt1 activity, and a gene carrier, the therapeutic effect of which can be achieved by bringing the gene therapeutic agent into contact with pulmonary tumor cells. Examples of the effectors useful in the present invention include, but are not limited to, siRNA, an antisense molecule, an antagonist, a ribozyme, an inhibitor, a peptide and a small molecule with preference for siRNA.

A polyester amine is preferable as the gene carrier because of having high transfection efficiency.

For use as the contact, advantage may be taken of a liposome, a nanoliposome, a ceramide-containing nanoliposome, a proteoliposome, a nanoparticulate, a calcium phosphor-silicate nanoparticulate, a calcium phosphate nanoparticulate, a silicon dioxide nanoparticulate, a nanocrystalline particulate, a semiconductor nanoparticulate, poly(D-arginine), a nanodendrimer, a virus, calcium phosphate nucleotide-mediated nucleotide delivery, electroporation, microinjection, and aerosol delivery. Particularly, when a polyester amine is used as a carrier, aerosol delivery is preferred in terms of efficiencies regarding gene delivery and expression.

It is advantageous if the effector is in the form of a complex with the gene carrier because a complex is more resistant to external damage such as DNA degradation than the effector itself is. An example of such a complex is a polyester amine/siRNA structure.

In accordance with another aspect, the present invention pertains to a pharmaceutical composition for the prevention and treatment of lung cancer comprising the gene therapeutic agent as an active ingredient, plus a carrier. The carrier may be selected from a group consisting of a liposome, a nanoliposome, a ceramide-containing nanoliposome, a proteoliposome, a nanoparticulate, a calcium phosphor-silicate nanoparticulate, a calcium phosphate nanoparticulate, a silicon dioxide nanoparticulate, a nanocrystalline particulate, a semiconductor nanoparticulate, poly(D-arginine), a nanodendrimer, a virus, and calcium phosphate nucleotide.

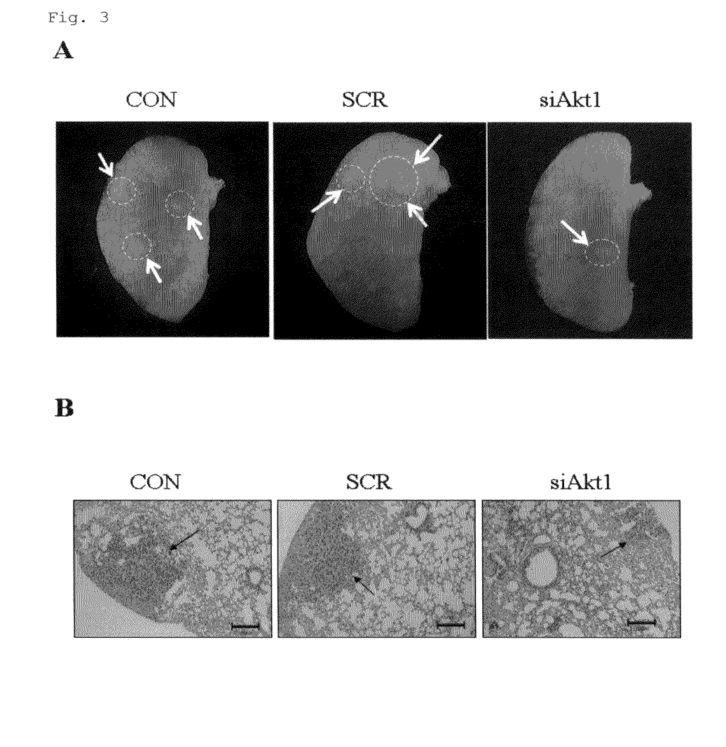

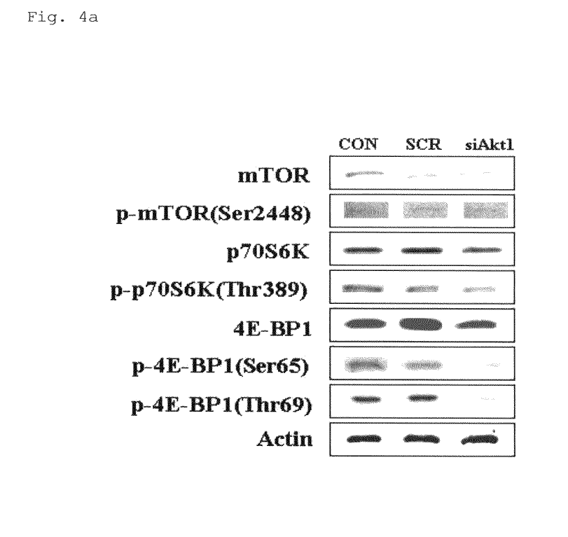

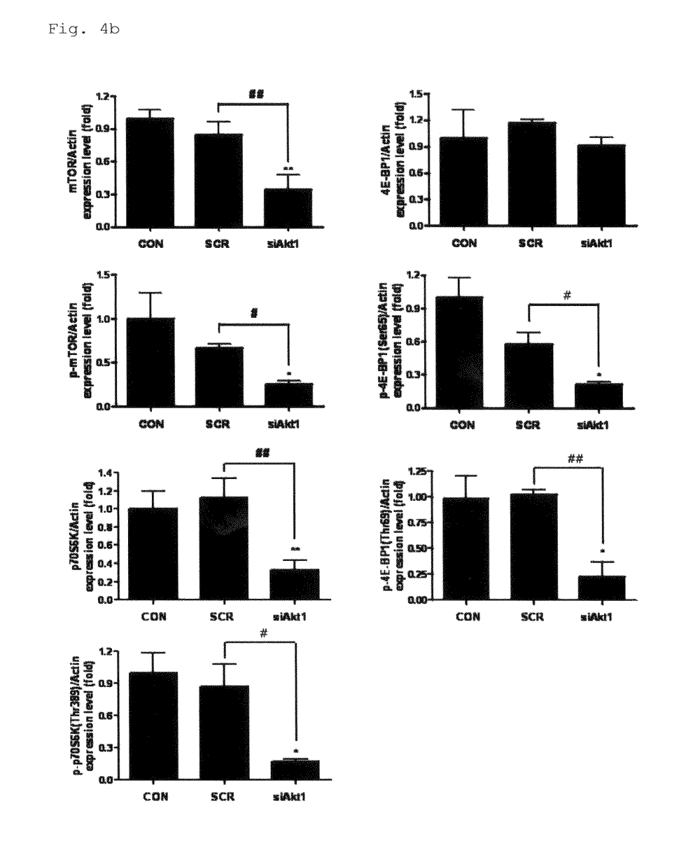

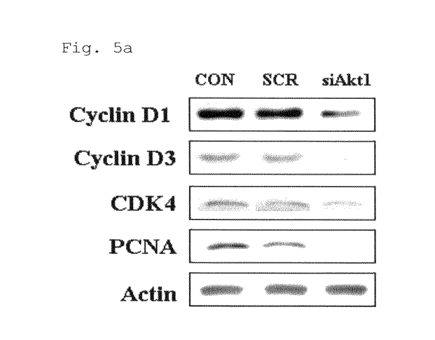

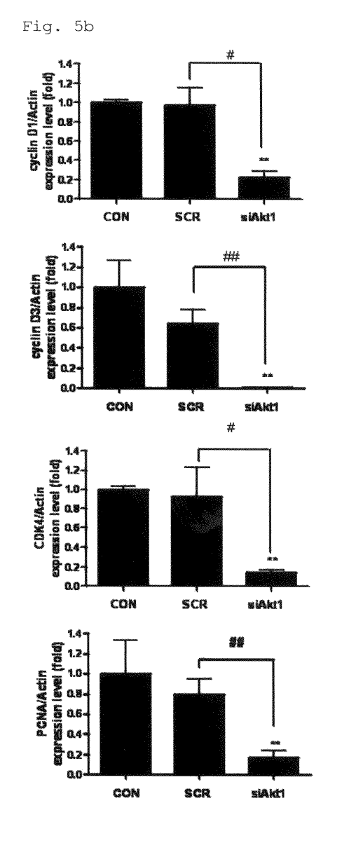

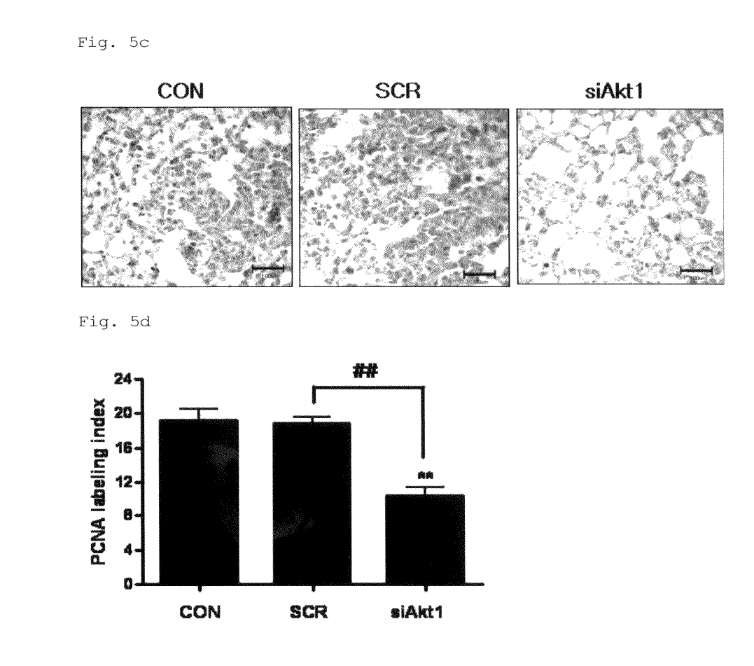

Here, the present inventors prove that the aerosol delivery of poly(ester amine)/Akt1 siRNA complexes, as will be explained in the following Examples, can suppress lung tumorigenesis in K-ras null lung cancer model mice through altering Akt signals and the cell cycle. In detail, it is demonstrated that knocking-out of Akt1 activity (but not the knocking-out of either Akt2 nor Akt3) is sufficient to suppress Akt1-related signals important for protein translation (FIG. 4) and cell cycle progression (FIG. 5), and thus inhibit pulmonary tumor progression (FIG. 3 and Table 1) in K-ras null mice.

In consequence, it is demonstrated that Akt1 knockout is sufficient to delay tumorigenesis onset and to provide profound resistance to K-ras gene mutation-induced lung tumor development. A battery of recent evidence has also demonstrated that Akt1 deficiency was sufficient to significantly attenuate the tumor development induced by PTEN (phosphatase and tensin homolog deleted on chromosome 10) deficiency (Chen et al., 2006).

Also provided was strong evidence that the aerosol delivery of PTEN suppressed Akt downstream pathways in the lungs of K-ras null mice (Kim et al., 2004). A recent report also showed that partial Akt1 ablation could be used as a general approach to inhibit tumorigenesis induced by lesions which do not directly activate Akt (Skeen et al., 2006).

Interestingly, it was reported that Akt1 knockout mice were not impaired in their lifespan and might possibly live longer than wild-type mice (Chen et al., 2006). Notably, it was recently suggested that Akt1 suppresses metastasis (Wyszomierski and Yu, 2005). These results suggest that partial ablation of Akt could be used as a therapeutic approach for cancer without eliciting severe physiological consequences.

In the last decade, many studies have focused on the correlation between cell cycle control and lung carcinogenesis. Just as apoptosis is controlled by highly conserved machinery, cell cycle is also a highly conserved mechanism by which eukaryotic cells proliferate.

In the present invention, the effect of aerosol-delivered Akt1 siRNA on cell cycle control in lungs of K-ras null mice was investigated and was found to suppress the lung cancer growth through inhibiting the cell proliferation proteins such as cyclins D1 and D3, CDK4 and PCNA (FIG. 5). The initiation of cell cycle control via extracellular signals induces the transcription of several proteins, including cyclin D, which, when complexed with CDK4, moves into the next cell cycle (Caputi et al., 2005).

These results are supported by the findings that Akt1 is associated with cyclin D1 up-regulation, which contributes to the disruption of the G1/S regulatory point of the cell cycle and leads to abnormal cell proliferation during carcinogenesis (Parekh and Rao, 2007).

Consequently, the aerosol-delivered poly(ester amine)/Akt1 siRNA complex efficiently suppressed lung cancer progression through regulating proteins important for Akt-related signals and regulating the cell cycle. Taken together, the results of the present invention also strongly suggest that aerosol gene delivery may provide an effective noninvasive model of gene delivery and Akt1 siRNA may be effective for lung cancer prevention as well as treatment through its targeting of protein translation and cell cycle regulation.

The findings in this invention are summarized as follows.

First, the aerosol delivery of Akt1 siRNA is a promising approach for the treatment of lung cancer.

This study emphasizes the viability of developing effective and selective prophylactic options for lung cancer in light of extensive in vivo research into the therapeutic effects of Akt1 siRNA.

First, the aerosol delivery of Akt1 siRNA decreases the level of Akt1 protein particularly in the lungs.

Second, the aerosol delivery of Akt1 siRNA significantly suppresses lung tumorigenesis in K-ras null mice.

Third, the aerosol delivery of Akt1 siRNA inhibits proteins important for protein translation in lungs of K-ras null mice.

Finally, the aerosol delivery of Akt1 siRNA significantly inhibits proteins important for cell cycle regulation in lungs of K-ras null mice.

A better understanding of the present invention may be obtained through the following examples which are set forth to illustrate, but are not to be construed as limiting the present invention.

### Example 1

### Materials

Polyethylene imine (Mn 423, 97% purity) and poly(ethylene glycol)diacrylate (Mn 258, 97% purity) were purchased from Sigma-Aldrich (St. Louis, Mo., USA), and pcDNA3.1-green fluorescent protein (GFP) (6.1 kb) was purchased from Invitrogen (Carlsbad, Calif., USA). Monoclonal antibodies against Akt1 and phospho-Akt at Thr308 were produced using a general method described elsewhere.

Anti-phospho-Akt1 and anti-mTOR both at Ser473, and anti-pmTOR at Ser2448 were obtained from Cell Signaling Technology (Beverly, Mass., USA). Other antibodies for western blotting and IHC were purchased from Santa Cruz Biotechnology (Santa Cruz, Calif., USA).

### Construction of Akt1 siRNA and Preparation of Poly(Ester Amine)/siRNA Complex

Oligonucleotides encoding the 19-mer hairpin sequence of siRNA specific for Akt1 were designed using the method suggested by Meng et al. (2006). A scrambled siRNA which has the same nucleotide composition as the siRNA, but lacks significant sequence homology to the genome was also designed. These oligonucleotides were ligated into the sixpress Human U6 PCR vector system (Mirus Bio, Madison, Wis., USA) according to the manufacturer's instructions.

Poly(ester amine) was synthesized using a method known in the art (Park et al., 2005). The poly(ester amine)/siRNA complex at a charge ratio of 45 was chosen as the most efficient condition for gene delivery on the basis of previous results (Park et al., 2005). Briefly, the preparation of self-assembled poly(ester amine)/siRNA complex was initiated in distilled water by dropwise adding 1 mg of plasmid DNA to poly(ester amine) with gentle vortexing, and the final volume was adjusted to 50 ml. The complex was then incubated at room temperature for 30 minutes before use.

### Example 2

### In Vivo Aerosol Delivery of Poly(Ester Amine)/siRNA Complex

Experiments were performed on 5-week-old ICR mice and K-ras null mice. ICR mice were purchased from Joongang Laboratory Animal (Seoul, Korea) and K-ras null mice were obtained from the Human Cancer Consortium-National Cancer Institute (Frederick, Md., USA). The animals were kept in the laboratory animal facility with temperature and relative humidity maintained at 23±2° C. and 50±20%, respectively, under a 12-hour light/dark cycle. All methods used in these experiments were approved by the Animal Care and Use Committee at Seoul National University (SNU-061110-5). For gene delivery, mice were placed in the nose-only exposure chamber and exposed to the aerosol based on the methods used previously (Tehrani et al., 2007; Kim et al., 2004).

For the test of gene delivery efficiency of poly(ester amine), ICR mice were divided into three groups of three mice each. Two groups were exposed to aerosol containing GFP plasmid DNA with or without poly(ester amine), respectively, while the other group was used as a control. Two days after exposure, these mice were sacrificed and the lungs were collected for the detection of GFP green signal.

To determine the effects of Akt1 siRNA on lung cancer development, separately, the K-ras null mice were divided into three groups of four. The control group was not treated with anything and the other two groups were exposed to aerosol containing poly(ester amine) with Akt1 siRNA or scrambled siRNA (scrambled control), respectively. The K-ras null mice were exposed to aerosol twice a week for a total of 4 weeks. At the end of the test period, the K-ras null mice were killed, and the lungs were harvested.

During the autopsy procedure, the neoplastic lesions of lung surfaces were carefully counted. Simultaneously, the lungs were perfused and fixed in 10% neutral buffered formalin for histopathologic examination and immunohistochemistry (IHC). The lungs from four mice were used for histopathologic and immunohistochemical analysis. The remaining lungs were stored at −70° C. for further study.

### Example 3

### Western Blot Analysis

For Western blot analysis, the lungs of three among four mice were selected by random sampling. After measuring the protein concentration of homogenized lysates using a Bradford kit (Bio-Rad, Hercules, Calif., USA), 30 μg protein was separated on sodium dodecyl sulfate-polyacrylamide electrophoresis gel and transferred to nitrocellulose membranes.

The membranes were blocked for 1 hour in (TTBS) containing 5% skim milk, followed by immunoblotting by incubating the membranes overnight with their corresponding primary antibodies in 5% skim milk at 4° C., and then with secondary antibodies conjugated to horseradish peroxidase (HRP) for 3 hours at room temperature or overnight at 4° C.

After washing, bands of interest were analyzed by the luminescent image analyzer LAS-3000 (Fujifilm, Tokyo, Japan), and the quantification of Western blot analysis was done using the Multi Gauge version 2.02 program (Fujifilm, Tokyo, Japan).

### Example 4

### Histopathologic Analysis and Immunohistochemistry (IHC)

The lung tissues were fixed in 10% neutral buffered formalin, embedded in paraffin, and sectioned at 4 μm thickness. For histologic analysis, the tissue sections were stained with hematoxylin and eosin. For IHC, the tissue sections were deparaffinized in xylene and rehydrated through alcohol gradients, then washed and incubated in 3% hydrogen peroxide (Appli-Chem, Darmstadt, Germany) for 30 minutes to quench endogenous peroxidase activity.

After being washed in PBS, the tissue sections were incubated with 5% bovine serum albumin in PBS for 1 hour at room temperature to block unspecific binding sites. Primary antibodies were applied to tissue sections overnight at 4° C. On the following day, the tissue sections were washed and incubated with secondary HRP-conjugated antibodies (1:50) for 1 hour at room temperature. After careful washing, the tissue sections were counterstained with Mayer's hematoxylin (Dako, Carpinteria, Calif., USA) and washed with xylene. Cover slips were mounted using Permount (Fisher, Pittsburgh, Pa., USA), and the slides were observed under a light microscope (Carl Zeiss, Thornwood, N.Y., USA).

The evaluation of phospho-Akt staining at Ser473 and Thr308 was done according to the scoring system of Tang et al. (2006), and the evaluation of PCNA staining was conducted as described by Zhang et al. (2000).

### RESULTS AND DISCUSSION

First, poly(ester amine) is an effective carrier for aerosol gene delivery

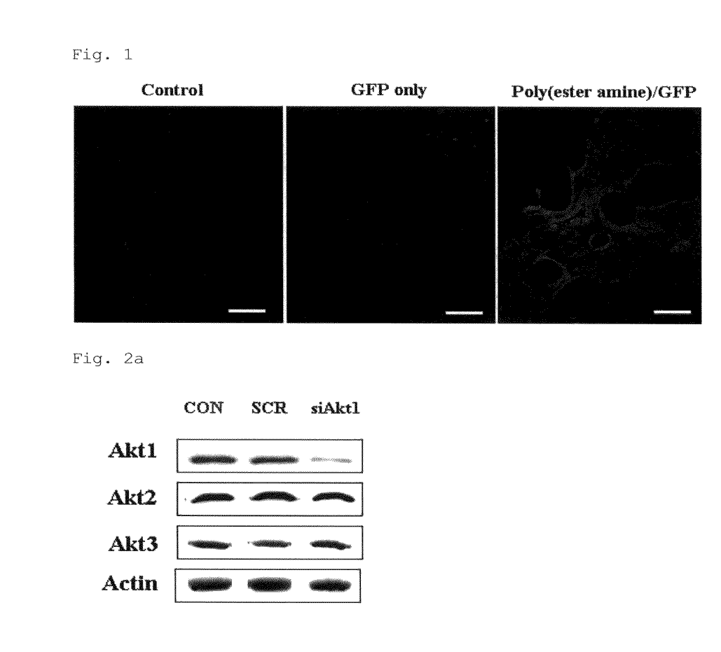

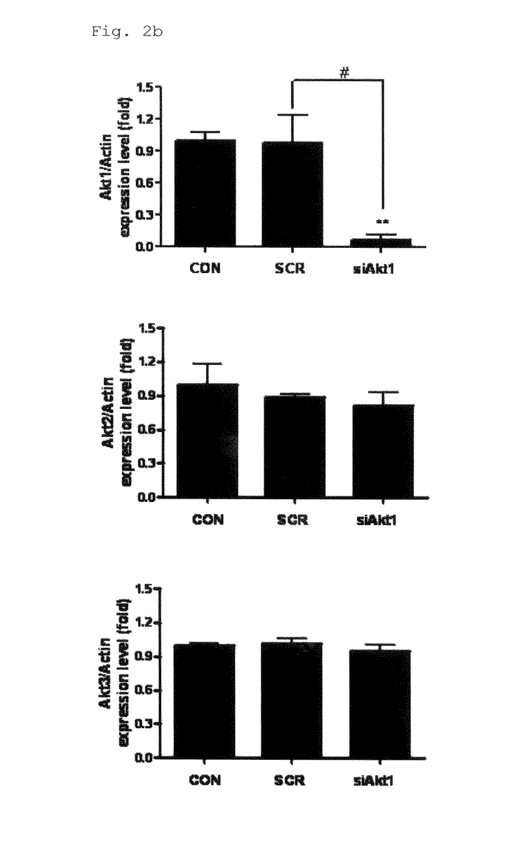

It was confirmed that in vivo transfection efficiency of poly(ester amine) in the lungs of ICR mice was high as found using a gene structure producing green fluorescent protein (GFP). When the mice were exposed to aerosol-delivered polyester amine carrying the siRNA, the green fluorescent signal of green fluorescent protein (GFP) was dominant in the poly(ester amine)/GFP complex-exposed group compared with the other two groups (FIG. 1), indicating that the delivery system of the present invention functioned efficiently.

Second, the aerosol delivery of Akt1 siRNA suppresses the expression of Akt1 protein particularly in lungs.

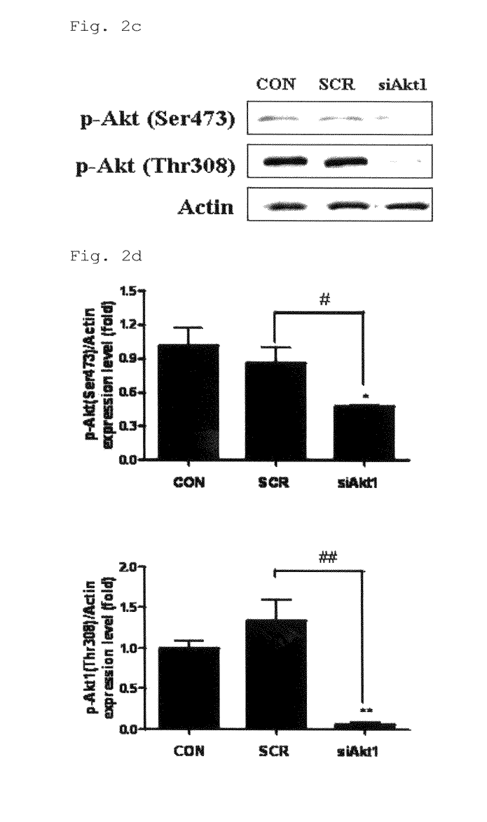

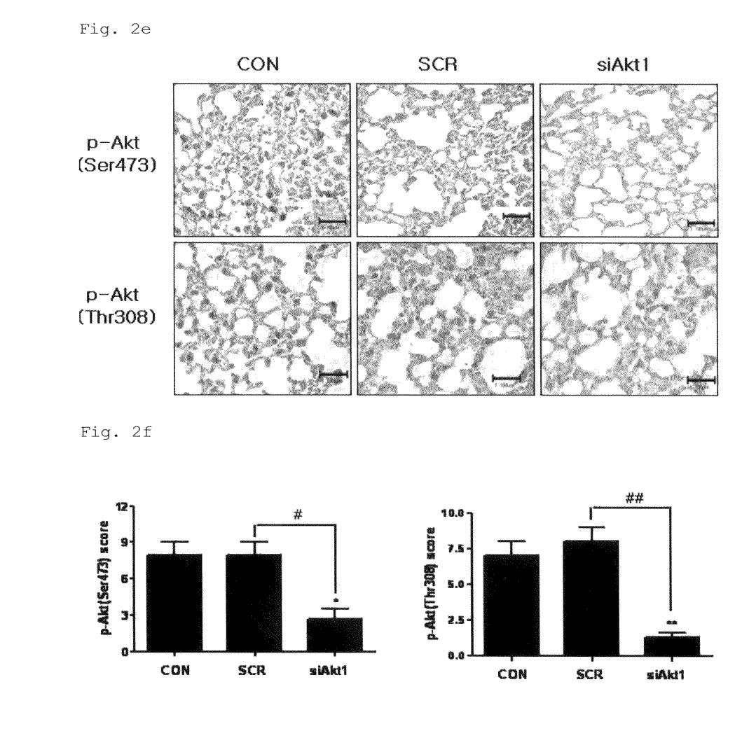

To determine whether aerosol-delivered Akt1 siRNA might particularly target Akt1, the mRNA and protein expressions of Akt1, Akt2, and Akt3 were measured by Western blot of the lungs of the mice exposed to the aerosol-delivered Akt1 siRNA. As shown in FIGS. 2a and 2b, aerosol-delivered Akt1 siRNA suppressed the mRNA and protein expression of Akt1 specifically without affecting the Akt2 and Akt3 in the lungs of K-ras null mice.

In consideration of the fact that Akt requires phosphorylation of both Thr308 and Ser473 for full activity (Tehrani et al., 2007; West et al., 2003), the phosphorylation status of Akt1 was examined in the lungs of K-ras null mice. Akt1 siRNA significantly inhibited the phosphorylation of Akt at Ser473 as well as Thr308 (FIG. 2c). Moreover, densitometric analysis explained a significant decrease in Akt phosphorylation at the critical sites (FIG. 2d).

Such suppressed Akt1 phosphorylation was further confirmed by IHC as shown in FIG. 2e, and also by counts of immunopositive cells as shown in FIG. 2f.

Third, the aerosol delivery of Akt1 siRNA significantly suppresses lung tumorigenesis in K-ras null mice.

To examine whether a decrease in Akt1 level changed the development pattern of tumorigenesis in the lung cancer model, the Akt1-derived siRNA was carried to the lung of K-ras null mice by aerosol polyester amine. The K-ras null mice were exposed to aerosol twice a week for a total of four weeks.

As shown in FIG. 3a (arrows and circles), the number of tumors and the mean tumor diameter were significantly decreased by Akt1 siRNA. Histopathologic examination also indicated that pulmonary tumor formation was significantly suppressed (arrows in FIG. 3b).

The range of tumorigenesis suppression is summarized in Table 1, below.

TABLE 1

| | | |
|---|---|---|
| | | hyperplasia |
| No. of | Adenoma | incidence |

| Groups | No.^{a} | Tumor^{b} | incidence^{c} | +++^{d} | ++^{e} |
|---|---|---|---|---|---|
| CON | 4 | 18 ± 0.3 | 3 | 2 | 0 |
| SCR | 4 | 18 ± 0.5 | 2 | 1 | 1 |
| siAkt1 | 4 | 11 ± 0.5*^{#} | 1 | 0 | 2 |

^{a}numbers of mice experimented
^{b}numbers of tumors in mice
^{c}numbers of the mice observed to be disabled
^{d}moderate class of atypical alveolar epithelial hyperplasia
^{e}mild class of atypical alveolar epithelial hyperplasia
*considerably different from control, with significance of p < 0.05.
^{#}considerably different from SCR, with significance of p < 0.05.

As apparent from the data of Table 1, Akt1 siRNA decreased adenoma incidence, and suppressed the generation of hyperplasia as well as the number of tumors.

Fourth, the aerosol delivery of Akt1 siRNA inhibits proteins important for protein translation in lungs of K-ras null mice.

The Akt activated by phosphorylation plays a central role in tumorigenesis. The Akt/mTOR pathway controls cellular protein translation through the regulation of p70S6K and 4E-BP1 phosphorylation, and protein translation closely related with cancer cell growth (Lawlor and Alessi, 2001). To obtain mechanistic insight into how Akt1 siRNA suppresses lung tumorigenesis, the proteins important for Akt-related protein translation signals were analyzed. The results showed that the inhibition of Akt1 significantly decreased mTOR and phospho-mTOR protein expressions.

Also, the aerosol delivery of Akt1 siRNA suppressed the protein expression of p70S6K and phosphor-p70S6K. The Akt1 siRNA suppressed phosphorylation at Ser65 as well as Thr69 of 4E-BP1 in lungs of K-ras null mice significantly, but did not affect the protein expression of total 4E-BP1 (FIGS. 4a and 4b).

Fifth, the aerosol delivery of Akt1 siRNA significantly inhibits proteins important for cell cycle regulation in lungs of K-ras null mice.

Akt is known to regulate cell cycle progression (Lawlor and Alessi, 2001). Accordingly, Akt1 siRNA was evaluated for effect on cell cycle-regulated proteins in the lungs of K-ras null mice. The results demonstrated that the aerosol delivery of Akt1 siRNA suppressed the proteins important for cell cycle regulation, such as cyclin D1, cyclin D3, CDK4, and PCNA (FIGS. 5a and 5b).

The expression of PCNA, a marker of cell proliferation, was further analyzed. Results were obtained by IHC (FIG. 5c) and by PCNA labeling index of the immunopositive cells (FIG. 5d). From these results, it was clearly indicated that Akt1 siRNA suppressed cell proliferation in the lungs of K-ras null mice.

As described above, the polyester amine/Akt1 siRNA complex according to the present invention can be delivered to the lungs of K-ras null mice through a nose-only inhalation system. The aerosol delivery of polyester amine-mediated Akt1 siRNA is provided as an effective model for noninvasive gene therapy because it can significantly suppress lung cancer progression.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A gene therapeutic for prevention or treatment of lung cancer, comprising an effector for suppressing Akt1 activity, and a gene carrier, by being brought into contact with pulmonary tumor cells,
wherein the effector is selected from a group consisting of siRNA, an antisense molecule, an antagonist, a ribozyme, an inhibitor, a peptide and a small molecule;
the gene carrier is polyester amine; and
the contact is conducted with the aid of a means selected from a group consisting of a liposome, a nanoliposome, a ceramide-containing nanoliposome, a proteoliposome, a nanoparticulate, a calcium phosphor-silicate nanoparticulate, a calcium phosphate nanoparticulate, a silicon dioxide nanoparticulate, a nanocrystalline particulate, a semiconductor nanoparticulate, poly(D-arginine), a nanodendrimer, a virus, calcium phosphate nucleotide-mediated nucleotide delivery, electroporation, microinjection, and aerosol delivery.

2. The gene therapeutic as set forth in claim 1, wherein the effector is siRNA.

3. The gene therapeutic as set forth in claim 1, wherein the effector forms a complex with the gene carrier.

4. The gene therapeutic as set forth in claim 3, wherein the complex is of a polyester amine/siRNA structure.

5. The gene therapeutic as set forth in claim 1, wherein the contact is conducted by aerosol delivery.

6. A pharmaceutical composition for prevention and treatment of lung cancer, comprising the gene therapeutic of claim 1 as an active ingredient, and a carrier.

7. The pharmaceutical composition as set forth in claim 6, wherein the carrier is selected from a group consisting of a liposome, a nanoliposome, a ceramide-containing nanoliposome, a proteoliposome, a nanoparticulate, a calcium phosphor-silicate nanoparticulate, a calcium phosphate nanoparticulate, a silicon dioxide nanoparticulate, a nanocrystalline particulate, a semiconductor nanoparticulate, poly(D-arginine), a nanodendrimer, a virus, and calcium phosphate nucleotide.

8. A pharmaceutical composition for prevention and treatment of lung cancer, comprising the gene therapeutic of claim 2 as an active ingredient, and a carrier.

9. A pharmaceutical composition for prevention and treatment of lung cancer, comprising the gene therapeutic of claim 3 as an active ingredient, and a carrier.

10. A pharmaceutical composition for prevention and treatment of lung cancer, comprising the gene therapeutic of claim 4 as an active ingredient, and a carrier.

11. A pharmaceutical composition for prevention and treatment of lung cancer, comprising the gene therapeutic of claim 5 as an active ingredient, and a carrier.
